(19) United States
(12) Patent Application Publication
(10) Pub. No.: US 2006/0058213 A1
(43) Pub. Date: Mar. 16, 2006

(54) Anti-viral and anti-bacterial cleaning composition

(75) Inventor: Christopher Malyszewicz, Bugbrooke (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Greenbridge Environmental Control Limited, Lewins Mead (GB)

(21) Appl. No.: 11/054,474

(22) Filed: Feb. 9, 2005

Related U.S. Application Data
(63) Continuation of application No. PCT/GB04/02148, filed on May 17, 2004.
(63) Continuation of application No. PCT/GB03/03296, filed on Jul. 30, 2003.

(30) Foreign Application Priority Data
May 15, 2003 (GB) GB 0311174.7

(51) Int. Cl. C11D 3/37

(52) U.S. Cl. 510/499

(57) ABSTRACT

According to one feature of the present invention there is provided a composition comprising at least one alcohol, at least one long-chain alkyl polyamine. According to a further feature of the present invention there is provided a composition comprising at least one long-chain alkyl polyamine and at least one halogen. Both compositions being suitable for application to a surface and substantially all of the microbial contamination.

This application is a continuation of PCT/GB2004/002148 filed May 17, 2004 designating the United States, and a continuation of PCT/GB2003/03296 filed July 30, 2003 designating the Unites States and claims priority under 35USC 119 to GB 0311174.7 filed May 15, 2003.

### FIELD OF INVENTION

The present invention relates to a liquid cleansing composition having an anti-viral and/or anti-bacterial action. More particularly, but not exclusively, it relates to a surface cleansing composition having both anti-viral and anti-bacterial activity.

### BACKGROUND

There is an ever expanding number of household products such as handwashes and domestic cleaning sprays professing to provide anti-bacterial properties. Often these products claim to eliminate work surfaces and the like of all known bacteria. Such claims are typically misleading at best. Widely reported research has shown that many of these currently available products are no better at reducing the onset of coughs, colds or other such infections or ailments than thoroughly washing one hands or cleaning the work surfaces. Indeed many such infections or aliments are caused by viruses which currently available anti-bacterial products are unable to combat, despite what they purport to achieve.

There is also an increasing concern about bacterial and viral infections being transmitted to patients and staff in hospitals and the like. One vector of infection is believed to be incompletely disinfected surfaces, which may harbour bacteria and/or viruses that are resistant to existing surface cleaning agents. There is a strong suspicion that the spread of the recent SARS (Severe Acute Respiratory Syndrome) outbreak may have been linked to the ability of the SARS virus to resist conventional cleaning agents/disinfectants. Viruses spread from an infected patient thus remain viable and ready to be picked up by and to infect other patients and medical staff. Other pathogens, such as the MRSA bacterium, are also suspected to be surviving existing surface cleaning/disinfecting agents and routines.

It is known to use cationic surfactants, such as quaternary ammonium salts, as dual-purpose surface cleaning agents and bactericides. However, while such materials are generally found to be sufficient to deal with, say, food-poisoning bacteria in a food preparation environment, they are not regarded as sufficiently active to handle more dangerous and more resistant pathogens in a medical context.

Alcohols, such as iso-propanol, and halogens, such as iodine, have in the past been used individually as relatively crude disinfecting agents around wounds and skin lesions, but they have not proven suitable for wide area cleaning of hard surfaces and the like. For example, iodine can stain many surfaces, and its use at high concentrations is limited by safety considerations.

It is an object of the present invention to provide a liquid cleansing and disinfecting preparation, suitable for use on hard surfaces, with a high anti-viral and anti-bacterial effectiveness.

### SUMMARY OF INVENTION

According to one feature of the present invention there is provided a composition comprising at least one alcohol and at least one long-chain alkyl polyamine.

According to a further feature of the present invention there is provided a composition comprising at least one long-chain alkyl polyamine and at least one halogen.

The present invention may be a composition for cleaning and disinfecting. Such a composition may be an aqueous surface cleaning and disinfecting composition.

The present invention may alternatively or additionally be an anti-bacterial composition and/or an anti-viral composition. Furthermore, the present invention may alternatively or additionally be an anti-fungal composition.

According to a further feature of the present invention, there is provided a means of destroying organisms and/or inhibiting the ability of bacteria and/or viruses to replicate when said bacteria and/or viruses are present on a surface, the means comprising the application of a composition to said surface wherein the composition is configured to rupture the phospholipid membrane of the bacteria or virus, the composition being further configured to substantially permanently bind to bacterial DNA and viral DNA or RNA. In this context “substantially permanently” is understood to mean that the bacterial DNA or viral DNA or RNA has a component or components of the composition bound thereto such that said DNA or RNA is unable to replicate for at least several hours, but preferably indefinitely. These means advantageously allow for rapid decontamination of a surface. Preferably the composition comprises at least one alcohol and at least one long-chain alkyl polyamine. Alternatively the composition preferably comprises at least one long-chain alkyl polyamine and at least one halogen.

According to a further aspect of the present invention, there is provided a means of inhibiting the ability of bacteria and/or viruses to replicate when said bacteria and/or viruses are present on a surface, the means comprising the application of a composition to said surface wherein the composition is configured to substantially permanently encapsulate the bacterial or viral structures and prevent the replication of their genetic material. In this context “substantially permanently” is understood to mean that the bacteria or virus has a component or components of the composition bound thereto such that said DNA or RNA is encapsulated to the degree that it is unable to replicate for at least several hours, but preferably indefinitely. These means advantageously allow for rapid decontamination of a surface. Preferably the composition comprises at least one alcohol and at least one long-chain alkyl polyamine. Alternatively the composition preferably comprises at least one long-chain alkyl polyamine and at least one halogen.

In the various aspects of the present invention, the long-chain alkyl polyamine compound preferably comprises a long-chain alkyl triamine compound and/or a long-chain alkyl tetramine compound.

The composition may comprise a mixture of long-chain alkyl polyamine compounds having a range of different alkyl chain lengths.

Advantageously, the long-chain alkyl polyamine compound may comprise a compound of the general formula R—NH—(CH₂)ₘ—NH—(CH₂)ₙ—NH₂, where R is a linear or branched alkyl chain comprising at least eight carbon atoms, and each of m and n may equal either 2 or 3.

R may be a linear or branched alkyl chain comprising between ten and fourteen carbon atoms. Preferably R is a linear alkyl chain, and each of m and n may equal 3.

The composition preferably comprises between 10% and 30% by volume of the long-chain alkyl polyamine compound or compounds.

Advantageously, the composition comprises between 15% and 25% of the long-chain alkyl polyamine compound or compounds. Optionally, the composition may comprise 20%±2% of the long-chain alkyl polyamine compound or compounds.

In the applicable various aspects of the present invention, the at least one aliphatic alcohol preferably comprises between one and four carbon atoms. These compositions preferably comprise two aliphatic alcohols. It is particularly preferred that these compositions comprise ethanol and n-propanol.

These compositions may comprise between 10% and 30% by volume aliphatic alcohols. Advantageously, these compositions comprise between 15% and 25% by volume aliphatic alcohols. These compositions may comprise between 10% and 20% by volume ethanol and between 5% and 10% by volume n-propanol. Optionally, these compositions may comprise between 14% and 16% by volume ethanol and between 5% and 7% by volume n-propanol.

In the applicable various aspects of the present invention, the compositions preferably comprise a mixture of halogens and/or halogen source(s). Alternatively, these compositions may comprise only a single halogen and/or single halogen source. The preferred halogen of the applicable aspects of the present invention is iodine, the preferred source of iodine being molecular iodine provided in a solid form.

These compositions preferably comprise up to 0.5% by weight iodine. Advantageously, the composition may comprise between 0.1 % and 0.5% by weight iodine. Optionally, these compositions may comprise 0.33%±0.05% by weight iodine. These compositions of may comprise a complexing agent adapted to form a complex with the halogen.

The compositions of the present invention may comprise at least one buffering agent, such as nitrilotriacetic acid or its salts.

The compositions of the present invention may comprise at least one surfactant. Preferably at least one amphoteric surfactant is present in the composition. Ideally a mixture of amphoteric surfactants are present in the composition.

The compositions of the present invention may comprise at least one wetting agent, such as a polyglycol ether, optionally a polyethylene glycol ether or a polypropylene glycol ether.

The compositions of the present invention are preferably non-dangerous. In this context “non-dangerous” is understood to mean non-dangerous as defined by European Dangerous Preparations Directive (99/45/EC) and the Dangerous Substances Directive (67/548/EEC).

Although the compositions of the present invention are effective in a non-diluted form, there are numerous applications where a diluted composition would be desirable. The composition is preferably configured to be effective for a range of dilutions. For instance, the range for which the composition may be effective can be at any level of dilution with water up to a 1:1000 parts of water dilution. Normally the lower limit of dilution is a 1:1 dilution with water, but preferably the range of dilution for which the composition is effective is 1:5 parts of water to 1:100 parts of water dilution. The most preferred dilution however, is substantially 1 part of composition to substantially 9 parts of water.

The compositions of the present invention may be provided in a form which is suitable for a number of different forms of delivery to a surface. The composition could be provided in a concentrated form for subsequent dilution by a user shortly before being used to clean a surface. Once diluted however, the resultant solution may be capable of being stored for up to 12 months and yet still being effective against bacteria and/or viruses and/or fungus and/or a provide a conventional detergent/cleansing effect which removes macroscopic soiling.

The compositions of the present invention may be provided in a form ready for immediate delivery to a surface. The delivery device for such immediate delivery of the composition may be a controlled spray, such as a trigger spray or the like. A trigger spray advantageously allows a user to have a degree of remoteness from the surface the composition is to be used on, such that the composition may have already started to attack the bacteria and/or viruses present on said surfaces by the time the user comes into contact with the surface. Preferably the compositions for immediate delivery are capable of being stored in its delivery device for up to 24 months and yet still being effective against bacteria and/or viruses and/or fungus and/or a provide a conventional detergent/cleansing effect which removes macroscopic soiling.

Another delivery device for immediate delivery of the compositions of the present invention may be an impregnated cloth wipe. Such wipes could be provided in a container or drum containing numerous wipes, or provided in a single sachet form. Preferably such wipes are capable of being stored in their container for up to 24 months and yet still being effective against bacteria and/or viruses and/or fungus and/or a provide a conventional detergent/cleansing effect which removes macroscopic soiling.

According to a further aspect of the present invention there is provided a method for manufacturing a composition for cleaning and disinfecting wherein the method comprises: the addition to a pH buffered solution of at least one long-chain alkyl polyamine, to which is added at least one surfactant to make an interim solution; separate to said interim solution a premix solution containing at least one alcohol is made; the interim solution and the premix solution are then combined.

According to a further aspect of the present invention there is provided a method for manufacturing a composition for cleaning and disinfecting wherein the method comprises: the addition to a pH buffered solution of at least one long-chain alkyl polyamine, to which is added at least one surfactant to make an interim solution; separate to said interim solution a premix solution containing at least one halogen is made; the interim solution and the premix solution are then combined.

### BRIEF DESCRIPTION OF THE DRAWINGS

FIG. 1 shows the chemical structure of the long-chain alkyl polyamine of the present invention.

### DETAILED DESCRIPTION

In order to allow the present invention to be more readily understood embodiments of the invention will now be described more particularly by way of example only, and with reference to the accompanying drawings in which:

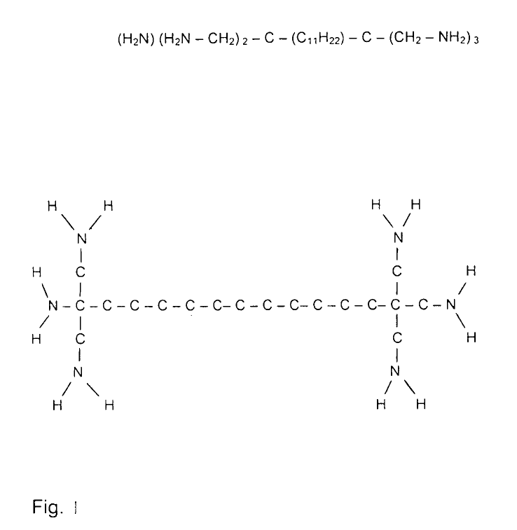

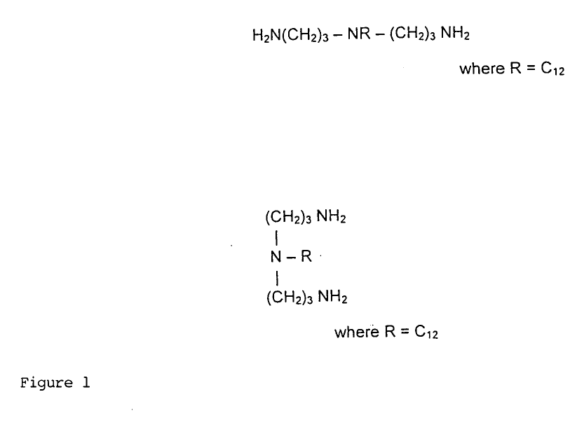

FIG. 1 shows the chemical structure of the long-chain alkyl polyamine of the present invention.

### EXAMPLE 1

An aqueous surface cleaning composition, comprising:

| | |
|---|---|
| NTA 89% powder | 0.85 kg |
| Ethanol | 15.0 litres |
| n-Propanol | 6.0 litres |
| Topanol O FG | 0.55 litres |
| Sandoteric SC | 2.42 litres |
| Sandozin NRW conc | 6.95 litres |
| Sandoteric ABD | 4.45 litres |
| Triameen Y12D-30 | 19.99 litres |
| Deionised water | 43.4524 litres |

### EXAMPLE 2

An aqueous surface cleaning composition, comprising:

| | |
|---|---|
| NTA 89% powder | 0.85 kg |
| Topanol O FG | 0.55 litres |
| Sandoteric SC | 2.42 litres |
| Sandozin NRW conc | 6.95 litres |
| Sandoteric ABD | 4.45 litres |
| Triameen Y12D-30 | 19.99 litres |
| Deionised water | 43.4524 litres |
| Iodine (solid) | 0.3376 Kg |

NTA is nitrilotriacetic acid trisodium salt, a buffering agent. Topanol O FG is food-grade butylated hydroxytoluene, an antioxidant, sold by Chance & Hunt Ltd. (Topanol is a registered trade mark of ICI plc). Sandozin NRW conc is a polyethoxylate ether sold by Clariant as a wetting agent. It also forms a relatively stable complex with iodine. Sandoteric SC is a sulphobetaine amphoteric surfactant, which acts as a detergent, and Sandoteric ABD is a complex mixture of amphoteric surfactants acting as a detergent and having a degree of bactericidal activity. Both are sold by Clariant. (Sandozin and Sandoteric are registered trade marks of Novartis SA).

Triameen Y12D-30 is a long-chain alkyl triamine of the general formula R′—NH—C₃H₆—NH—C₃H₆—NH₂, where R′ is a “tallow alkyl”—a naturally-derived mixture of alkyl chains of different lengths, the most common of which is a dodecyl chain. It is sold by Akzo Nobel.

FIG. 1 illustrates the hypothesised structure of the long-chain alkyl triamine. For Example 1, it is possible that the activity of this molecule is located on the NH₂ groups, where these groups are accessible toward DNA or RNA. The nucleotides of the DNA or RNA may then be liganded by addition to phosphatide groupings by the presence of this amine group.

Alternatively, for Example 2, it is possible that the activity of this molecule is also located on the NH₂ groups, wherein the iodine is monotonically bonded to the nitrogen yet accessible toward DNA or RNA. The nucleotides of the DNA or RNA may then be liganded by addition to phosphatide groupings by the presence of the iodated amine group. Thus, the iodine radical may be free to roam on the molecule and as there is partial addition thereof, there is competition for valencey fulfilment.

In any event, although the mechanism of attack on bacterium for either of Example 1 or Example 2 is not fully understood, it is expected that in a suitably buffered solution the long-chain alkyl triamine forms a cationic species. Together with the surfactant(s), preferably amphoteric in nature, the triamine attacks the phospholipid membranes which form the outer wall of a bacterium. In most cases, these membranes are ruptured or lysed, leading to release of the bacterium's DNA. The triamine and the surfactant(s) are believed to attack bacterial DNA and bind to critical parts of the helix preventing it from replicating. The alcohol(s) may also contribute to the attack on the membranes.

Even where the membranes are not sufficiently damaged to release their contents for destruction, the composition is adapted to inactivate the bacterium for prolonged periods (at least 14 days in current testing, much longer than for current cleaners/disinfectants).

Turning to the mechanism of attack on viruses, similarly this mechanism is also not fully understood. However it is again expected that the cationic triamine, formed in a suitably buffered solution, attacks the outer wall of the capsid of the virus in conjunction with the surfactant(s), which are preferably amphoteric in nature. It is possible that these structures are ruptured or lysed as a consequence of the attack, leading to release of the viral DNA or RNA. The complexed halogen, for Example 2, or the alcohol(s), for Example 1, are believed to act in conjunction on viral DNA or by bonding or associating themselves with parts of the viral RNA.

Additionally the triamine and the surfactant(s) are also believed to attack the viral DNA or RNA by binding to critical parts of the helix. The result of the attack(s) on the viral DNA or RNA is the inhibition of the DNA or RNA's ability to replicate. For Example 1, the alcohol(s) may also contribute to the attack on the membranes, particularly the outer viral phospholipid envelope, present in some but not all DNA/RNA viruses.

Alternatively, rather than the viral capsid being ruptured or lysed by the attack of the buffered cationic triamine and the surfactant(s), it is possible that the attack results in the binding to surface structures, blocking and inactivating viral receptors. The result of this attack being the inhibition of infectivity, thus preventing the virus spreading to other cells. It is possible that halogen, for Example 2, or the alcohol(s), for Example 1, take some part in the attack of the viral capsid membrane.

Regardless of the mechanism, the combined action of the components of the composition of Example 1 or Example 2, the result is the break up and destruction of a majority of the organism and/or the inhibition of any viruses or bacteria for prolonged periods. The composition may also have a conventional detergent/cleansing effect, removing macroscopic soiling from a surface to which it is applied, as well as washing off inhibited bacteria or viruses as well as the associated debris of the destroyed organism. The composition should have minimal deleterious effect on the surfaces tested.

The composition of Example 2 would not stain surfaces as would conventional formulations containing similar levels of halogen, particularly iodine.

As already mentioned, the mechanism of virucidal action by a composition of the present invention is not clearly understood. From the constituents present in the composition, it is suspected that the quaternary ammonium compound(s) bind to anionic phosphate groups and fatty acid chains in phospholipids. It is also suspected that the alcohol of Example 1 would denature proteins. Both mechanisms damage the microbial membranes. It is further suspected that the halogen of Example 2 may modify structural proteins and may inhibit enzymes through halogenation of amino acids in proteins.

Through experimentation, various naked virus or purified animal deoxy-ribose nucleic acid (DNA) samples can be treated with the compositions described by the present invention. An interaction takes place, so that when placed in an electric field under gel electrophoresis, a DNA smear is produced instead of the expected DNA ladder of normal integrity, indicating alteration of the ionisation characteristics of DNA. If the same composition/virus or composition/animal DNA mixture is extracted with a mixture of phenol/chloroform, the composition itself is broken down, and the full DNA electrophoretic pattern will be restored with normal integrity. This data indicates that the DNA (viral or animal DNA) is not degraded during the treatment with the composition, but that the composition interaction with viral or animal DNA alters the normal DNA structural and ionic integrity.

The interaction between the compositions described by the present invention can be further investigated for its affect on viral particles and viral ribose nucleic acid (RNA). For example, where the composition (1 part in 10 parts water) as 9 part diluted composition and 1 part poliovirus vaccine (final 1000 copies /ml) for periods of 5, 15, 30 and 60 minutes, followed by extraction, using QIAGEN silicon columns (QIAGEN incorporates a protease step for protein degradation), prior to complimentary deoxy-ribose nucleic acid (cDNA) synthesis and DNA amplification, (with detection of nucleic acid product in a in-house real-time Lightcycler quantitative RNA assay), reductions in the RNA viral load should be seen from 1000 copies to 50, 50, 10, and 10 copies respectively-(reductions of 95%, 95%, 99%, 99%), compared to 1000, 1000, 1000, 1000 copies/ml respectively in water control samples. Such an experiment can be repeated with composition/virus incubations of 5, 15, 30 and 60 minutes, as previously stated, but RNA extraction performed with a phenol/chloroform procedure (once with phenol, once with 1:1 phenol and chloroform, and once with chloroform rather than by the QIAGEN extraction method), RNA detection should be detected at 1000, 1000,1000, and 1000 copies respectively (no reduction in RNA load).

Such experimentation should demonstrate that the composition does not degrade RNA over the periods of 1-60 minutes, but that an interaction occurs between the composition and poliovirus/poliovirus RNA. This interaction inhibits protease action (active in the QIAGEN process) to cleave composition peptides that would normally release RNA, or alters naked RNA ionically, so that RNA cannot then be further captured and amplified in the test. The result would be a low recovery of RNA, and will appear as a low copy number in the assay. The effect of the composition-virus interaction should be removed during the chemical extraction with phenol. It can be concluded that the composition is not destructive to viral capsids, but inhibits enzymatic cleavage of the capsid, and requires further stringent chemical extraction to achieve release of the nucleic acid. The QIAGEN effect referred to here is a consistent effect and could be replicated for other RNA and DNA viruses including Adenovirus, BK virus and Norovirus, and appears to be a plausible mechanism of virucidal activity, whereby the altered viral structure is resistance to physiological and enzymatic attack.

It can therefore be concluded that both viral DNA and viral RNA are not degraded by treatment with the compositions described by the present invention. However, the compositions' interactions with the viral capsid prevent enzymatic cleavage. Although the process of virus uncoating involves different mechanisms for different virus groups, they all require the viral capsid to have structural and ionic integrity and be susceptible to cellular physiological processes that allow it to pass into and through the host cell, and interact with cellular structures and enzyme systems. The changes induced by the composition on viral structures are likely to prevent this process and account for its virucidal activity.

The composition also has a degree of activity against fungi, moulds and yeasts, although it is believed that a modified formulation might be required for full effectiveness against the tougher walls of fungal spore cells and the like.

The compositions described by the present invention pass the standard “555-challenge” test (see British Standard BS EN 1276:1997 and the French Afnor test). As an effective anti-viral and anti-bacterial cleansing agent, it may be categorised as a (2) category disinfectant in the system employed by the UK National Health Service, suitable for cleaning in “medium high risk” areas.

1. A composition comprising at least one alcohol and at least one long-chain alkyl polyamine.

2. A composition according to claim 1, wherein the composition is a cleaning and disinfecting composition.

3. A composition according to claim 1, wherein the composition is an aqueous surface cleaning and disinfecting composition.

4. A composition according to claim 1, wherein the composition is an anti-bacterial composition.

5. A composition according to claim 1, wherein the composition is an anti-viral composition.

6. A composition according to claim 1, wherein the composition is an anti fungal composition.

7. A composition according to claim 1, wherein the long-chain alkyl polyamine compound comprises a long-chain alkyl triamine compound and/or a long-chain alkyl tetramine compound.

8. A composition according to claim 1, wherein the composition comprises a mixture of long-chain alkyl polyamine compounds having a range of different alkyl chain lengths.

9. A composition according to claim 1, wherein the long-chain alkyl polyamine compound comprises a compound of the general formula H₂N(CH₂)₃—NR—(CH₂)₃—NH₂, where R is a linear or branched alkyl chain comprising at least eight carbon atoms.

10. A composition according to claim 9, wherein R may be a linear or branched alkyl chain comprising between ten and fourteen carbon atoms.

11. A composition according to claim 9, wherein R is a linear alkyl chain.

12. (canceled)

13. A composition according to claim 1, wherein the composition comprises between 10% and 30% by volume of the long-chain alkyl polyamine compound or compounds.

14. A composition according to claim 1, wherein the composition comprises between 15% and 25% of the long-chain alkyl polyamine compound or compounds.

15. A composition according to claim 1, wherein the at least one aliphatic alcohol comprises between one and four carbon atoms.

16. A composition according to claim 1, wherein the composition comprises two aliphatic alcohols.

17. A composition according to claim 1, wherein the composition comprises between 10% and 30% by volume aliphatic alcohols.

18. A composition according to claim 1, wherein the composition comprises between 15% and 25% by volume aliphatic alcohols.

19. A composition according to claim 1, wherein the composition comprises between 14% and 16% by volume ethanol and between 5% and 7% by volume n-propanol.

20. A composition according to claim 1, wherein the composition comprises at least one buffering agent.

21. A composition according to claim 1, wherein the composition comprises at least one surfactant.

22. A composition according to claim 21, wherein the at least one surfactant is amphoteric.

23. A composition according to claim 1, wherein the composition comprises at least one wetting agent.

24. A composition comprising at least one long-chain alkyl polyamine and at least one halogen.

25. A composition according to claim 24, wherein the composition is a cleaning and disinfecting composition.

26. A composition according to claim 24, wherein the composition is an aqueous surface cleaning and disinfecting composition.

27. A composition according to claim 24, wherein the composition is an anti-bacterial composition.

28. A composition according to claim 24, wherein the composition is an anti-viral composition.

29. A composition according to claim 24, wherein the composition is an anti-fungal composition.

30. A composition according to claim 24, wherein the long-chain alkyl polyamine compound comprises a long-chain alkyl triamine compound and/or a long-chain alkyl tetramine compound.

31. A composition according to claim 24, wherein the composition comprises a mixture of long-chain alkyl polyamine compounds having a range of different alkyl chain lengths.

32. A composition according to claim 24, wherein the long-chain alkyl polyamine compound comprises a compound of the general formula H₂N(CH₂)₃—NR—(CH₂)₃NH₂, where R is a linear or branched alkyl chain comprising at least eight carbon atoms.

33. A composition according to claim 24, wherein R may be a linear or branched alkyl chain comprising between ten and fourteen carbon atoms.

34. A composition according to any of claims 32, wherein R is a linear alkyl chain.

35. (canceled)

36. A composition according to claim 24, wherein the composition comprises between 10% and 30% by volume of the long-chain alkyl polyamine compound or compounds.

37. A composition according to claim 24, wherein the composition comprises between 15% and 25% of the long-chain alkyl polyamine compound or compounds.

38. A composition according to claim 24, wherein the composition comprises at least one buffering agent.

39. A composition according to claim 24, wherein the composition comprises at least one surfactant.

40. A composition according to claim 24, wherein the at least one surfactant is amphoteric.

41. A composition according to claim 24, wherein the composition comprises at least one wetting agent.

42. A composition according to claim 24, wherein the composition comprises a mixture of halogens and/or halogen source(s).

43. A composition according to claim 24, wherein the composition comprises only a single halogen and/or single halogen source.

44. A composition according to claim 24, wherein the halogen is iodine.

45. A composition according to claim 44, wherein the composition comprises up to 0.5% by weight iodine.

46. A composition according to claim 44, wherein the composition comprises 0.33%±0.05% by weight iodine.

47. A composition according to claim 24, wherein the composition comprises a complexing agent adapted to form a complex with the halogen.

48. A means of destroying bacteria and/or inhibiting the ability of bacteria and/or viruses to replicate when said bacteria and/or viruses are present on a surface, the means comprising the application of a composition to said surface wherein the composition is configured to rupture the phospholipid membrane of the bacteria or virus, the composition being further configured to cleave bacterial DNA and/or substantially permanently bind to bacterial DNA and viral DNA or RNA.

49. A means according to claim 48, wherein the composition of claim 1 or claim 24 is provided.

50. A means of inhibiting the ability of bacteria and/or viruses to replicate when said bacteria and/or viruses are present on a surface, the means comprising the application of a composition to said surface wherein the composition is configured to substantially permanently encapsulate the bacteria or virus and prevent the replication of their genetic material.

51. A means according to claim 50, wherein the composition of claim 1 or claim 24 is provided.

52. A method for manufacturing a composition for cleaning and disinfecting wherein the method comprises:
the addition to a pH buffered solution of at least one long-chain alkyl polyamine, to which is added at least one surfactant to make an interim solution;
separate to said interim solution a premix solution containing at least one alcohol is made; and the interim solution and the premix solution are then combined.

53. A method for manufacturing a composition for cleaning and disinfecting wherein the method comprises:
the addition to a pH buffered solution of at least one long-chain alkyl polyamine, to which is added at least one surfactant to make an interim solution;
separate to said interim solution a premix solution containing at least one halogen is made; and the interim solution and the premix solution are then combined.
